(19) United States
(12) Patent Application Publication
(10) Pub. No.: US 2019/0021813 A1
(43) Pub. Date: Jan. 24, 2019

(54) OFFSET SUPPORT BAR AND MOUNTING SLEEVE

(71) Applicant: Adept Surgical Products, LLC, Holly Springs, NC (US)

(72) Inventors: David Julian Gomez, Holly Springs, NC (US); William J. Koteles, Holly Springs, NC (US)

(21) Appl. No.: 16/042,011

(22) Filed: Jul. 23, 2018

Related U.S. Application Data
(60) Provisional application No. 62/536,183, filed on Jul. 24, 2017.

(51) Int. Cl. A61B 90/50; F16B 2/12; A61B 17/02

(52) U.S. Cl. CPC A61B 90/50; A61B 17/0218; F16B 2/12

(57) ABSTRACT

An offset support bar for use in a patient treatment environment includes: a lower linear bar portion extending along a first longitudinal axis; a central bar portion connected to the lower linear bar portion; and an upper linear bar portion connected to the central bar portion and extending along a second longitudinal axis, which is parallel to the first longitudinal axis. The central bar portion, at least in part, and the second longitudinal axis are laterally offset from the first longitudinal axis in opposite lateral directions. The central bar portion may include: a first outreach section connected to the lower linear bar portion; and a second outreach section that is connected to the upper linear bar portion and that crosses the first longitudinal axis. The outreach sections may extend diagonally relative to the longitudinal axes.

### CROSS-REFERENCE TO RELATED APPLICATION

This application claims the benefit of priority of U.S. provisional patent application No. 62/536,183, titled “Offset Support Bar and Mounting Sleeve,” filed on Jul. 24, 2017, which is incorporated herein in its entirety by this reference.

### TECHNICAL FIELD

The present invention relates generally to the field of bed-side support devices for use in hospitals and medical facilities, and more particularly to support bars for surgical retractor systems.

### BACKGROUND

A support bar can used to support medical or surgical procedure equipment above a patient bed, typically by mounting the support bar on a clamp that engages a bed-side rail. Support bars adapted for obese patients are not readily available. Some existing support bars may have an offset middle portion to accommodate bed or patient portions that extend laterally beyond the rail upon which the clamp is mounted in use. The upper linear portion of such a support bar extends along a common axis with the lower end of the support bar. Thus, the upper end of the support bar is positioned over and is aligned with the lower end of the support bar, with no horizontal or lateral offset therebetween. This is not advantageous with respect to positioning supported equipment above a patient bed, particularly where larger patients are concerned.

Surgical retractor assemblies are widely used, for example to maintain surgical incisions as open and to support and position medical device during surgical procedures. Many retractor systems, such as those of the prior art (U.S. Pat. No. 4,254,763, U.S. Pat. No. 6,808,493, and US2006008484351), utilize the surgical table's existing bed rail system to provide rigid and foundational support and are typically used during abdominal, thoracic, spine, and neurological surgeries. While retractor systems may be required to clamp onto a surgical table's bed rail system, vertical support bars can differ in size, and the design of the clamp required to hold and engage a bed rail structure can vary.

According to the NIH, more than two-thirds (68.8 percent) of adults are considered to be overweight or obese. More than one-third (35.7 percent) of adults are considered to be obese. More than 1 in 20 (6.3 percent) have extreme obesity. Almost 3 in 4 men (74 percent) are considered to be overweight or obese. The majority of all surgical tables made worldwide are only 19.5 inches to 21 inches wide, and thus the use of common bed rail accessories and existing retracting systems can be particular injurious to obese patients, as their body habitus (skin, adipose tissue, and extremities) are subject to compressive forces by typical retractor support bars. The existing retractor systems fail to account for the lateral contours of obese patients when they are placed on space limited tables. As a result it is very common for the arms to be displaced in an abducted (away from the patient's body less than 45 degrees) fashion to try to account for the straight or fixed vertical angle of current retractor posts. In a common scenario, a post is placed against the patient's body directly. Some providers will place some padding (foam or gel) between the post and the patient's skin, but even then, the compressive transmural pressure over periods of time, despite the padding, places the patient at risk for skin, vascular, or nerve injury.

As surgical procedures have evolved into more laparoscopic or robotic based platforms, it is fairly common to tuck the patient's arms to facilitate both the procedure and surgical access. In the unfortunate event of an emergency, it is common for a thoracic or abdominal retractor to be placed so the surgery can be converted to an open (thoracic or laparotomy) procedure so the emergency can be controlled (i.e., bleeding, perforation, etc.). When the arms are adducted (arm secured at sides), especially in obese patients, time is of the essence and the need for efficient retractor affixation is important, especially in hemorrhagic (bleeding) scenarios.

### SUMMARY

This summary is provided to introduce in a simplified form concepts that are further described in the following detailed descriptions. This summary is not intended to identify key features or essential features of the claimed subject matter, nor is it to be construed as limiting the scope of the claimed subject matter.

According to at least one embodiment, an offset support bar for use in a patient treatment environment includes: a lower linear bar portion extending along a first longitudinal axis; a central bar portion connected to the lower linear bar portion; and an upper linear bar portion connected to the central bar portion and extending along a second longitudinal axis, which is parallel to the first longitudinal axis. The central bar portion, at least in part, and the second longitudinal axis are laterally offset from the first longitudinal axis in opposite lateral directions.

The central bar portion may include: a first outreach section connected to the lower linear bar portion; and a second outreach section that is connected to the upper linear bar portion and that crosses the first longitudinal axis.

The central bar portion may further include a longitudinally extending intermediate section connected to the first outreach section and second outreach section.

The intermediate section may extend along a third longitudinal axis; and the third longitudinal axis and the second longitudinal axis may be offset from the first longitudinal axis in said opposite lateral directions.

The upper linear bar portion may be laterally offset from the first longitudinal axis in a first lateral direction; the intermediate section may be laterally offset from the first longitudinal axis in a second lateral direction; and the second lateral direction may be opposite the first lateral direction.

In at least one example, the first outreach section and second outreach section each extend diagonally relative to the first longitudinal axis.

The lower linear bar portion may include a longitudinal terminal end formed as a shank having a square cross section (for example having a one half inch side). The upper linear bar portion may include a longitudinal terminal end having a square cross section (for example having a three eighths inch side). A lateral side dimension of the terminal end of the lower linear bar portion may be different from a lateral side dimension of the terminal end of the upper linear bar portion.

The offset support bar may include an accessory mounting device, the mounting device including: an engagement bracket configured to releasably mount on the upper linear bar portion; and a tubular mounting sleeve extending from the engagement bracket.

The engagement bracket includes a clamp, and a handle operative to tighten and loosen the clamp.

The mounting device may include a lower end having a circular interior bore, and an intermediate section between the lower end and the tubular mounting sleeve, wherein the intermediate section of the mounting device has a square cross-sectioned interior bore.

In at least one example, the tubular mounting sleeve has a longitudinally extending circular interior bore.

The upper linear bar portion may include a longitudinal terminal end having a square cross section, and the square cross-sectioned interior bore of the intermediate section of the mounting device may be dimensioned to receive and non-rotatably engage the longitudinal terminal end of the upper linear bar portion.

The offset support bar may further include a depth-stop flange surrounding an upper end of the lower linear bar portion.

In at least one embodiment, a mounting device for use in a patient treatment environment includes: a tubular mounting sleeve; and an engagement bracket connected to the tubular mounting sleeve, the engagement bracket including a clamp, and a handle operative to tighten and loosen the clamp.

A series of mounting sleeves having internal bores varying in ½ inch increments may be provided to improve and account for different retracting systems or to accommodate any dimensional changes to retractors as a result of this general accessory utility. For example a 0.75 inch, a 1 inch, and a 1.5 inch mounting sleeve may each be provided so as to assure a universal adapter assembly is available.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated.

FIG. 1 shows a clamp and support bar according to the prior art

FIG. 2 is a perspective view of an improved offset support bar, according to at least one embodiment.

FIG. 3 is an elevation view of the offset support bar of FIG. 2.

FIG. 4 is a perspective view of an improved mounting sleeve according to at least one embodiment.

FIG. 5A is a cross-sectional view of the mounting sleeve of FIG. 4, taken at the line 5A-5A.

FIG. 5B is another cross-sectional view of the mounting sleeve of FIG. 4, taken at the line 5B-5B.

FIG. 6 is another perspective view of the mounting sleeve of FIG. 4.

FIG. 7 is an elevation view of the mounting sleeve of FIG. 4 mounted on the offset support bar of FIG. 2.

FIG. 8 is an elevation view of the combination of FIG. 7, shown coupled to a bed rail clamp according to at least one embodiment.

### DETAILED DESCRIPTIONS

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

Advantages of inventive embodiments described herein will allow some prior-existing surgical based retractor systems to be readily utilized on obese patient populations. Some existing retractor systems and their support accessories previously used during thoracic surgery could be supported by the support bar and mounting device described herein, in order to accommodate existing surgical retractor crossbars of one inch in diameter for example. Thus advantages include allowing cost restrictive healthcare systems to utilize existing infrastructure, and allowing a contoured support bar as described herein to be placed within a standard pole-receiving bed rail clamp adapter used in surgical procedures.

The use of a readily and contoured accessory will provide for a foundation of support, especially for existing one inch retractor systems, which would reduce the time of untucking the arm, placing in an abducted position (less than 45 degrees), and affixing the current straight bar design from minutes to mere seconds. The utility of a contoured accessory will provide for improved and expedient surgical care when caring for the obese patient population over current straight bar systems.

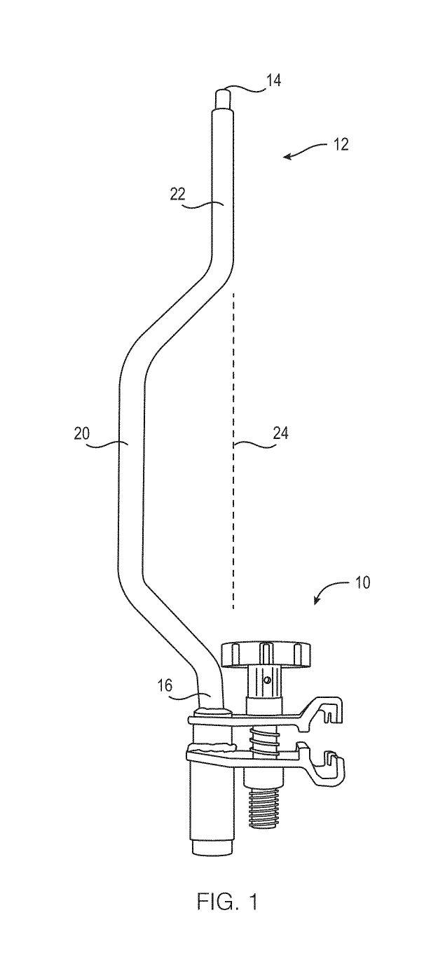

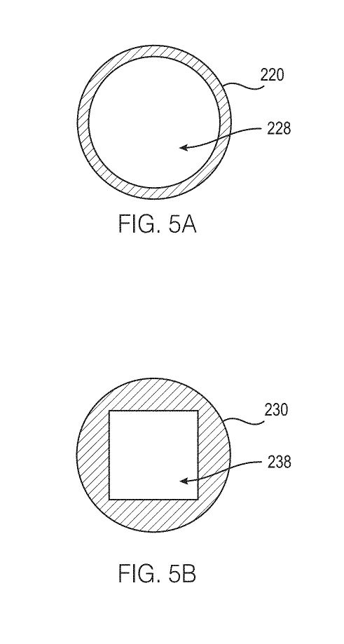

FIG. 1 shows a prior art clamp 10 and support bar 12, as may be used for example with a cross bar support system as shown in U.S. Pat. No. 6,387,047 (see crossbar support system 140 in FIG. 5 thereof). The support bar 12 can be used to support medical or surgical procedure equipment mounted upon the upper end 14 of the support bar. The lower end 16 of the support bar 12 is mounted onto the clamp 10, which can be engaged on a bed rail so as to support any equipment or devices mounted upon the upper end of the support bar above a patient during a medical procedure. The support bar 12 has an offset middle portion 20 to allow additional space in the surgical field near a patient.

As is typical in prior art support bars, an upper linear portion 22 of the support bar 12 extends along a common vertical axis 24 with the lower end 16 of the support bar 12. Thus, the upper end 14 of the support bar is positioned over and is aligned with the lower end 16, with no horizontal or lateral offset therebetween. The common axis 24 is described herein as vertical with respect to the typical use of the support bar 12.

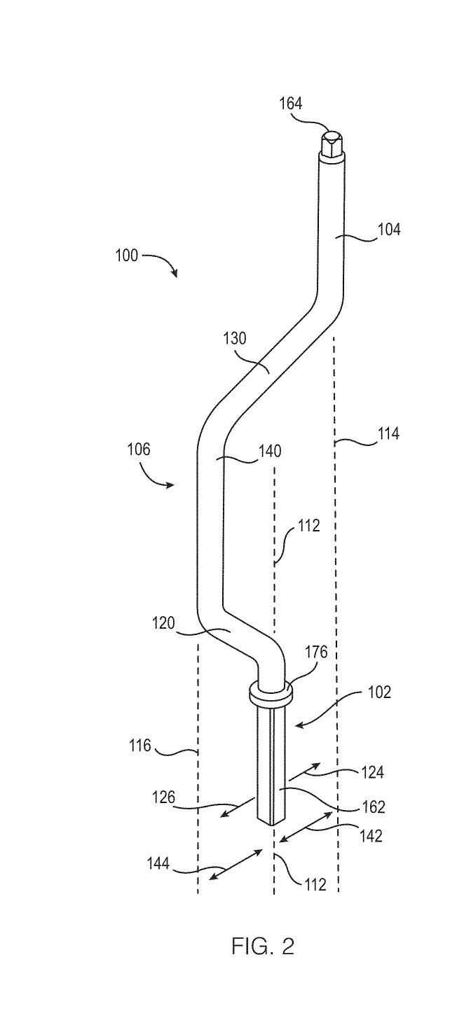

FIG. 2 shows an improved offset support bar 100, according to at least one embodiment. The support bar 100 has a lower linear bar portion 102 that extends along a first longitudinal axis 112, and an upper linear bar portion 104 that extends along a second longitudinal axis 114, which is generally parallel to the first longitudinal axis 112. The second longitudinal axis 114 is laterally offset from the first longitudinal axis 112. Accordingly, the upper linear bar portion 104 is laterally offset from the lower linear bar portion 102. When the offset support bar 100 is in use with the longitudinal axes 112 and 114 maintained as generally vertical, the upper linear bar portion 104 is horizontally offset from the lower linear bar portion 102. This arrangement is advantageous for placement of equipment over a patient when such equipment is mounted on the upper linear bar portion 104 during a medical procedure.

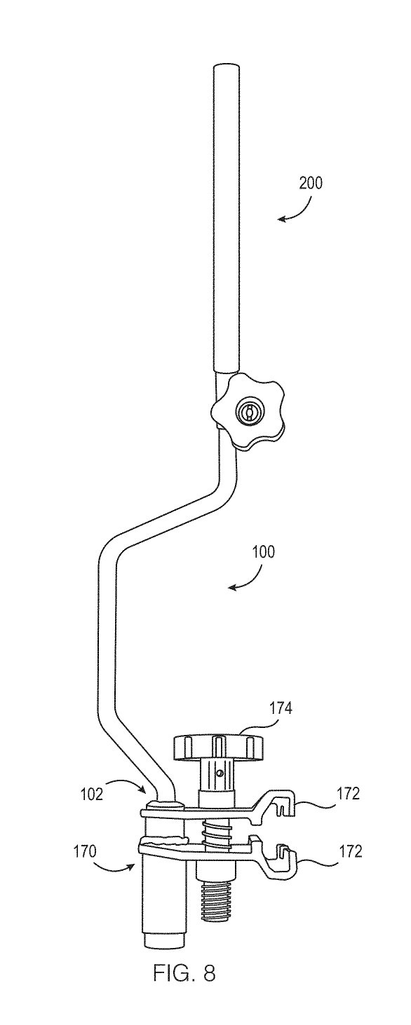

A central bar portion 106, which connects the lower bar portion 102 to the upper bar portion 104 is also at least in part laterally or horizontally offset from the lower linear bar portion 102, but offset laterally or horizontally opposite therefrom with respect to the upper linear bar portion 104. This arrangement is advantageous for accommodating patient size, comfort, and safety during a medical procedure given that portions of the patient's body or bedding may extend beyond a bed rail that supports the lower linear bar portion 102 when in use, for example by way of a clamp (see FIG. 8).

To provide such an arrangement, the central bar portion 106 includes a first outreach section 120 connected to the upper end of the lower linear bar portion 102. The first outreach section 120 extends away from the first longitudinal axis 112 toward a third longitudinal axis 116 that is generally parallel to and laterally offset from the first longitudinal axis 112. The third longitudinal axis 116 is offset from the first longitudinal axis in a lateral or horizontal direction 126, which is perpendicular to the longitudinal axes 112, 114 and 116. The second longitudinal axis 114 is offset from the first longitudinal axis 112 in a lateral or horizontal direction 124 that is opposite the direction 126. This arrangement is advantageous for both placement of equipment over a patient and for accommodating patient size, comfort, and safety.

The central bar portion 106 includes a second outreach section 130 connected to the lower end of the upper linear bar portion 104. The second outreach section 130 extends from the third longitudinal axis 116 to the second longitudinal axis 114. The second outreach section 130, which is longer than the first outreach section 120 in the illustrated embodiment, crosses the first longitudinal axis 112. The second outreach section 130 is connected to the first outreach section 120 such that the upper linear bar portion 104 is supported by the lower linear bar portion 102 when the offset support bar 100 is in use.

In the illustrated embodiment, the second outreach section 130 is connected to the first outreach section 120 by a longitudinally extending intermediate section 140 of the central bar portion 106. The intermediate section 140 extends along the third longitudinal axis 116 from a lower end of the intermediate section 140 that joins the first outreach section 120, to an upper end of the intermediate section 140 that joins the second outreach section 130.

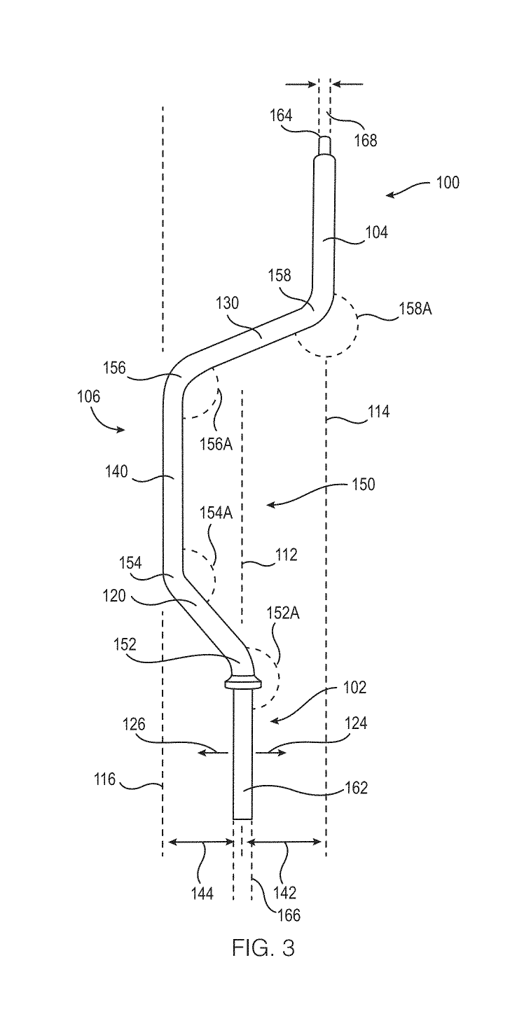

In the illustrated embodiment, the first outreach portion 120 extends diagonally (both longitudinally and laterally) relative the first longitudinal axis 112, and the second outreach section 130 extends diagonally relative to the second longitudinal axis 114 and first longitudinal axis 112. Thus, the angled joints 152, 154, 156 and 158 (FIG. 3) are non-binding and blunt (not sharp) from the patient side of the offset support bar 100, such that in use, the central bar portion 106 will route or hook partially around a patient while accommodating patient comfort and safety. In use, a patient, bedding, or other items may be positioned partially within the receiving area 150 defined between the first outreach portion 120 and second outreach section 130.

At the lowest angled joint 152, an exterior angle 152A that is greater than 180 degrees and less than 270 degrees is defined between the lower bar portion 102 and first outreach portion 120 from the patient side of the offset support bar 100. Similarly, at the highest angled joint 158, an exterior angle 158A that is greater than 180 degrees and less than 270 degrees is defined between the upper bar portion 104 and second outreach portion 130 from the patient side of the offset support bar 100. Thus, the angled joints 152 and 158 are blunt and thus permit movement of the patient, bedding and other materials or equipment items without damage, for example as they enter or exit the receiving area 150.

Within the interior of the receiving area 150, at the angled joint 154, an interior angle 154A that is less than 180 degrees and greater than 90 degrees is defined between the first outreach portion 120 and intermediate section 140. Similarly, at the angled joint 156, an interior angle 156A that is less than 180 degrees and greater than 90 degrees is defined between the intermediate section 140 and second outreach portion 130. Thus, the angled joints 154 and 156 are non-binding and thus permit movement of the patient, bedding and other materials or equipment items within the receiving area 150.

In the illustrated embodiment, the linear portions of the offset support bar 100 are formed as contiguous portions of a bar material and the angled joints 152, 154, 156 and 158 at the junctions of the linear portions are formed as bends. Other constructions and junction formations are within the scope of these descriptions.

Furthermore, in the illustrated embodiment, the lower two angled joints 152, 154 are formed as approximately 40 degree bends or junctions, such that the angles 152A and 154A are approximately 220 degrees and 140 degrees respectively. Also, in the illustrated embodiments, the upper two angled joints 156, 158 are formed as approximately 70 degree bends or junctions, such that the angles 156A and 158A are approximately 110 degrees and 250 degrees respectively.

A longitudinal terminal end 162 of the lower linear bar portion 102 of the offset support bar 100 in the illustrated embodiment is formed as a shank having a square profile or cross section if taken viewing along the first longitudinal axis 112. The lateral side dimension 166 (FIG. 3) of the terminal end 162 may be for example, one-half inch to match industry engagement standards. The lower linear bar portion 102 for example can engage standard bedrail clamps of the type having a square bore dimensioned to receive a one-half inch sided square cross-section bar.

A longitudinal terminal end 164 of the upper linear bar portion 104 of the offset support bar 100 in the illustrated embodiment is also formed as a shank having a square profile or cross section if taken viewing along the first longitudinal axis 112. The lateral side dimension 168 (FIG. 3) of the terminal end 164 may be, for example three-eighths inch to match industry engagement standards. The upper linear bar portion 104 for example can engage above-bed equipment and accessories.

Furthermore, in the illustrated embodiment, the remainder of the bar portion 104 below the square cross-sectioned terminal end 164 has a circular diameter, for example having a diameter of five eighths inch. In the illustrated embodiment, a depth-stop flange 176 (FIG. 2) surrounds an upper end of the lower linear bar portion 102 to prevent over insertion thereof into a host structure such as a bedrail clamp.

In at least one embodiment, the side dimension 166 (½ inch for example) of the terminal end 162 of the lower bar portion 102 and the side dimension 168 (⅜ inch for example) of the terminal end 164 of upper bar portion 104 are not matched so as to prevent misuse of the offset support bar 100 from its intended orientation with reference to upper and lower portions in these descriptions.

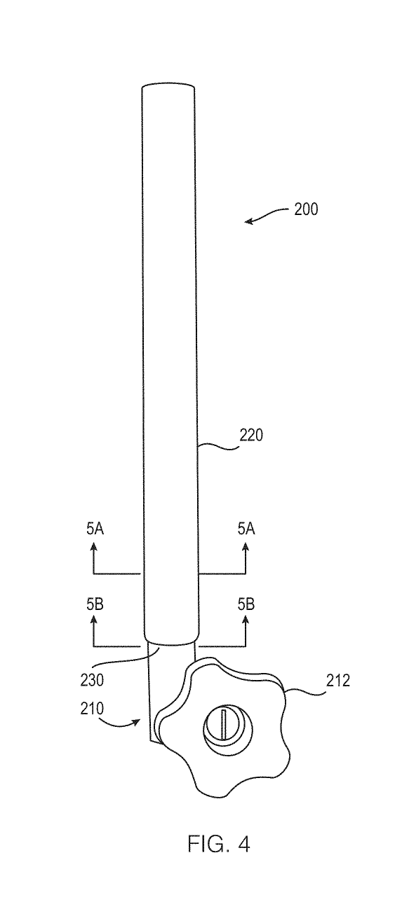

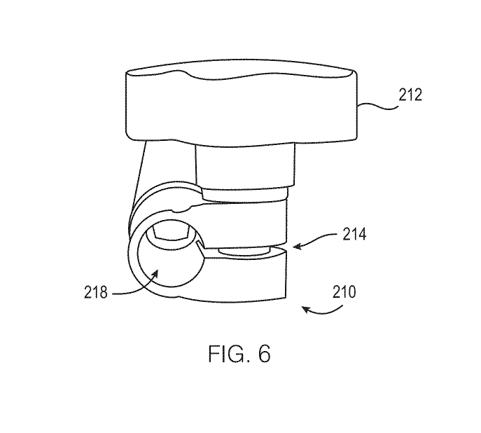

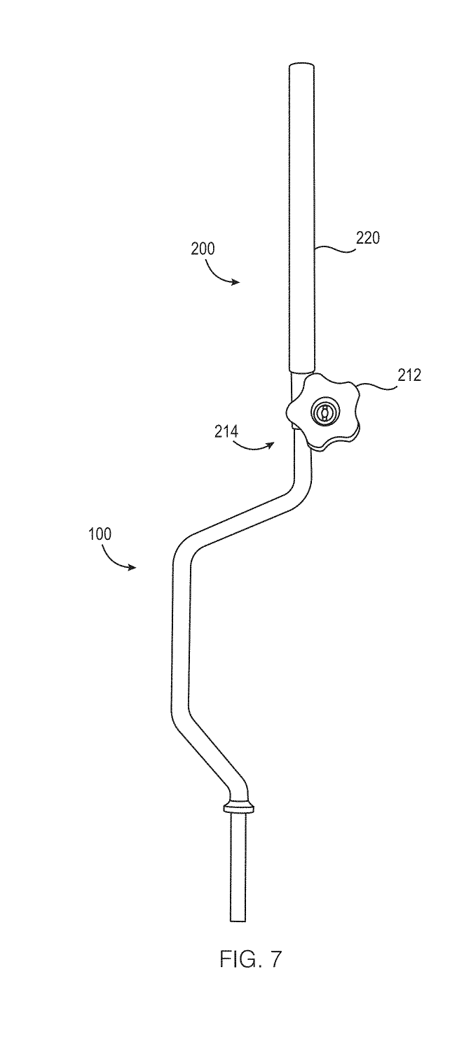

FIG. 4 is a perspective view of an improved mounting device 200 according to at least one embodiment. The mounting device 200 includes a lower engagement bracket 210 and an upper tubular mounting sleeve 220. The lower engagement bracket 210 has a rotatable handle 212, which is operative to loosen and tighten a clamp 214 as shown in FIG. 6. In at least one embodiment, the lower engagement bracket 210 at its lower end has a longitudinally extending circular interior bore 218, which is dimensioned (for example ⅝ inch plus tolerance) when open to loosely receive the circular diameter portion of the upper linear bar portion 104 of FIGS. 2-3. Thus, the mounting device 200 can be mounted upon the upper end of the offset support bar 100 as shown in FIG. 7. The handle 212 can be rotated to operatively: open the clamp 214 so as to permit installation and removal of the mounting device 200; and to close the clamp 214 so as to lock the mounting device 200 upon the offset support bar 100.

The upper tubular mounting sleeve 220 (FIG. 4) extends longitudinally, which is vertical in use, to receive and support additional equipment. The interior of the upper tubular mounting sleeve 220 has a generally circular cross section interior bore 228 as shown in FIG. 5A, and may have for example a five-eighths inch interior diameter to match industry engagement standards.

An intermediate section 230 (FIG. 4) of the mounting device 200 has an interior square cross section interior bore 238 (FIG. 5B), for example to receive and non-rotatably engage the terminal end 164 of the upper linear bar portion 104 of the offset support bar 100 of FIGS. 2-3. Thus, when mounted as shown in FIG. 7, the square cross section interior bore 238 (FIG. 5B) receives and engages the square cross section upper the terminal end 164 of the offset support bar 100, preventing their relative rotation.

In FIG. 8, the mounting device 200 is installed upon the offset support bar 100, which has the lower linear bar portion 102 thereof received by a bedrail clamp 170, which can be engaged on a bed rail so as to support any equipment or devices mounted upon the upper end of the mounting device 200 during a medical procedure. The clamp 170 has laterally extending jaws 172 between which a bedrail or other host structure can be captured to mount the entire illustrated assembly. A handle 174 can be rotated to open the jaws 172 so as to permit installation of the illustrated assembly; and to close the jaws 172 so as to lock the clamp 170 upon a bedrail or other structure.

In use, the lateral or horizontal offset spacing 142 (FIGS. 2,3), between the lower linear bar portion 102 along the first longitudinal axis 112 and the upper linear bar portion 104 along the second longitudinal axis 114, in the patient-side lateral direction 124 can be used to extend supported equipment and devices to more convenient positions above a patient during surgical or other medical procedures. Due to the opposite lateral or horizontal offset spacing 144, between the lower linear bar portion 102 and the intermediate section 140, being in the opposite the direction 126 from the patient-side lateral direction 124, patient comfort and safety are also accommodated. The first outreach portion 120 extends outward and laterally away from a patient, bedding or other items within the receiving area 150, and the second outreach section 130 returns laterally inward, for example over the side of a patient. Thus a closer accommodation over the patient's body area is provided and adds an improvement in stability when equipment, such as a retractor assembly, is supported above and by the offset support bar 100.

Bed sizes, medical equipment, and patient anatomies vary, for example depending on the size of the patient. These variations can be accommodated by the lateral offset spacing 144 of the central portion 104, and equipment and devices can be supported inward over a bed or patient relative to the edge rail of the bed.

The offset support bar 100 and mounting device 200 can each be constructed, for example, of stainless steel or other durable material that is strong and can be sterilized for multiple uses.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

What is claimed is:

1. An offset support bar for use in a patient treatment environment, the offset support bar comprising:
a lower linear bar portion extending along a first longitudinal axis;
a central bar portion connected to the lower linear bar portion; and
an upper linear bar portion connected to the central bar portion and extending along a second longitudinal axis, which is parallel to the first longitudinal axis,
wherein the central bar portion, at least in part, and the second longitudinal axis are laterally offset from the first longitudinal axis in opposite lateral directions.

2. The offset support bar of claim 1, wherein the central bar portion comprises:
a first outreach section connected to the lower linear bar portion; and
a second outreach section that is connected to the upper linear bar portion and that crosses the first longitudinal axis.

3. The offset support bar of claim 2, wherein the central bar portion further comprises a longitudinally extending intermediate section connected to the first outreach section and second outreach section.

4. The offset support bar of claim 3, wherein:
the intermediate section extends along a third longitudinal axis;
the third longitudinal axis and the second longitudinal axis are offset from the first longitudinal axis in said opposite lateral directions.

5. The offset support bar of claim 3, wherein:
the upper linear bar portion is laterally offset from the first longitudinal axis in a first lateral direction;
the intermediate section is laterally offset from the first longitudinal axis in a second lateral direction; and
the second lateral direction is opposite the first lateral direction.

6. The offset support bar of claim 2, wherein the first outreach section and second outreach section each extend diagonally relative to the first longitudinal axis.

7. The offset support bar of claim 1, wherein the lower linear bar portion comprises a longitudinal terminal end formed as a shank having a square cross section.

8. The offset support bar of claim 7, wherein the upper linear bar portion comprises a longitudinal terminal end having a square cross section.

9. The offset support bar of claim 8, wherein a lateral side dimension of the terminal end of the lower linear bar portion is different from a lateral side dimension of the terminal end of the upper linear bar portion.

10. The offset support bar of claim 1, further comprising a mounting device, the mounting device comprising:
an engagement bracket configured to releasably mount on the upper linear bar portion; and
a tubular mounting sleeve extending from the engagement bracket.

11. The offset support bar of claim 10, wherein the engagement bracket comprises a clamp, and a handle operative to tighten and loosen the clamp.

12. The offset support bar of claim 10, wherein the mounting device comprises a lower end having a circular interior bore, and an intermediate section between the lower end and the tubular mounting sleeve, wherein the intermediate section of the mounting device has a square cross-sectioned interior bore.

13. The offset support bar of claim 12, wherein the tubular mounting sleeve has a longitudinally extending circular interior bore.

14. The offset support bar of claim 12, wherein the upper linear bar portion comprises a longitudinal terminal end having a square cross section, and wherein the square cross-sectioned interior bore of the intermediate section of the mounting device is dimensioned to receive and non-rotatably engage the longitudinal terminal end of the upper linear bar portion.

15. The offset support bar of claim 1, further comprising a depth-stop flange surrounding an upper end of the lower linear bar portion.

16. A mounting device for use in a patient treatment environment, the mounting device comprising:
a tubular mounting sleeve; and
an engagement bracket connected to the tubular mounting sleeve, the engagement bracket comprising a clamp, and a handle operative to tighten and loosen the clamp.

17. The mounting device of claim 16, wherein the mounting device comprises a lower end having a circular interior bore, and an intermediate section between the lower end and the tubular mounting sleeve, the intermediate section having a square cross-sectioned interior bore.

18. The mounting device of claim 17, wherein the tubular mounting sleeve has a longitudinally extending circular interior bore.
